(19) United States
(12) Patent Application Publication
(10) Pub. No.: US 2024/0374271 A1
(43) Pub. Date: Nov. 14, 2024

(54) OBSTRUCTION REMOVAL SYSTEM

(71) Applicant: MicroVention, Inc., Aliso Viejo, CA (US)

(72) Inventors: Grant Warden, Aliso Viejo, CA (US); Jeff Inlow, Aliso Viejo, CA (US); Kiet Lam, Aliso Viejo, CA (US)

(73) Assignee: MicroVention, Inc., Aliso Viejo, CA (US)

(21) Appl. No.: 18/292,285

(22) PCT Filed: Jul. 29, 2022
(86) PCT No.: PCT/US2022/074334

§ 371 Date: Jan. 25, 2024

Related U.S. Application Data
(60) Provisional application No. 63/227,669, filed on Jul. 30, 2021.

(51) Int. Cl. A61B 17/221; A61B 17/00; A61B 90/00

(52) U.S. Cl. CPC A61B 17/221; A61B 17/00234; A61B 90/39; A61B 2017/00238; A61B 2017/00292; A61B 2017/2212; A61B 2090/3966

(57) ABSTRACT

An obstruction removal system for capturing and removing obstructions, such as clots or other matter, from a vascular system. The obstruction removal system may include one or more engaging members connected to an elongated member, such as to a distal end of the elongated member. The engaging member(s) may have an asymmetric body shape and/or an asymmetric cell configuration between its proximal and distal portions, such as a “tear drop” shape. The engaging member(s) may be comprised of a plurality of struts. One or more radiopaque markers may be connected to at least one of the struts, which may be comprised of a “dog bone” shape. A mesh structure may be positioned internally or externally to the engaging member(s) to act as a filter. A support wire may be positioned internally to aid in propping open the engaging member(s). Where multiple engaging members are used, they may have different sizes.

### RELATED APPLICATIONS

This application claims priority to U.S. Provisional Application Ser. No. 63/227,669 filed Jul. 30, 2021 entitled Obstruction Removal System, which is hereby incorporated herein by reference in its entirety.

### BACKGROUND OF THE INVENTION

Described herein are devices used to capture and remove obstructions, such as clots or other matter, from the vascular system, and systems and methods related to the delivery of such devices to a target area within the vascular system.

The buildup of thrombi in vasculature can lead to formation of blood clots. The formation of clots can result in restricted blood supply to downstream areas of the vasculature. When located in the neurovascular system, these clots can lead to stroke.

There is a need for an obstruction removal device which reduces the likelihood of fragmented thrombi remaining in the vasculature while maximizing the chance of mechanically capturing the clot while limiting the risk of endothelial denudation caused by high friction between the device and a vessel wall.

### BRIEF DESCRIPTION OF THE DRAWINGS

These and other aspects, features and advantages of which embodiments of the invention are capable of will be apparent and elucidated from the following description of embodiments of the present invention, reference being made to the accompanying drawings, in which

FIG. 1 is a side view of an engaging member according to an example embodiment.

FIG. 2 is a side view of an engaging member according to an example embodiment.

FIG. 3A is a side view of a plurality of connected engaging members according to an example embodiment.

FIG. 3B is a side view of a plurality of connected engaging members according to an example embodiment.

FIG. 4 is a flattened view of an engaging member strut pattern according to an example embodiment.

FIG. 5 is a flattened view of an engaging member strut pattern according to an example embodiment.

FIG. 6 is a magnified view of a structure for retaining a radiopaque marker according to an example embodiment.

FIG. 7 is a magnified view of a structure for retaining a radiopaque marker according to an example embodiment.

FIG. 8 is a magnified view of a radiopaque marker according to an example embodiment.

FIG. 9 is a magnified view of a radiopaque marker according to an example embodiment.

FIG. 10 is a magnified view of a radiopaque marker according to an example embodiment.

FIG. 11 is a magnified view of a radiopaque marker on a distal tip of an engaging member according to an example embodiment.

FIG. 12 is a side view of a step for creating the radiopaque marker of FIG. 11 according to an example embodiment.

FIG. 13 is a side view of a step for creating the radiopaque marker of FIG. 11 according to an example embodiment.

FIG. 14 is a side view of a step for creating the radiopaque marker of FIG. 11 according to an example embodiment.

FIG. 15 is a side view of a step for creating the radiopaque marker of FIG. 11 according to an example embodiment.

FIG. 16 is a side view of an engaging member with a mesh structure within it according to an example embodiment.

FIG. 17 is a side view of an engaging member with a mesh structure within it according to an example embodiment.

FIG. 18 is a side view of an engaging member with a mesh structure in between multiple engaging members according to an example embodiment.

FIG. 19 is a side view of an engaging member with a mesh structure engaging member behind it according to an example embodiment.

FIG. 20 is a side view of a mesh structure according to an example embodiment.

FIG. 21 is a side view of a mesh structure according to an example embodiment.

FIG. 22 is a side view of a mesh structure according to an example embodiment.

FIG. 23 is a side view of an engaging member with a support wire within it according to an example embodiment.

FIG. 24 is a side view of an engaging member with a support wire within it according to an example embodiment.

FIG. 25A is a side view of a first step for creating an engaging member according to an example embodiment.

FIG. 25B is a side view of a second step for creating an engaging member according to an example embodiment.

FIG. 25C is a side view of a third step for creating an engaging member according to an example embodiment.

### SUMMARY OF THE INVENTION

Disclosed herein is an obstruction removal device which reduces the likelihood of fragmented thrombi remaining in the vasculature while maximizing the chance of mechanically capturing the clot while limiting the risk of endothelial denudation caused by high friction between the device and a vessel wall.

In one example embodiment, an obstruction removal device may include an elongated member which is connected at or near its distal end to one or more engaging members. The one or more engaging members may have a collapsed configuration when sheathed and/or constrained, such as within a delivery device, and an expanded configuration when unsheathed and/or unconstrained.

In one example embodiment, only one engaging member may be either directly or indirectly connected to an elongated member, such as to a distal end of the elongated member. In another example embodiment, a plurality of engaging members (e.g., two, three, four, five, six, seven, or more engaging members) may be either directly or indirectly connected to the elongated member. In embodiments with multiple engaging members, only one, or more than one, of the engaging members may be connected to the elongated member.

In one example embodiment, one or more engaging members may be disposed over and connected to a wire or distal portion of the elongated member. In some example embodiments, the proximal and distal ends of each engaging member may be connected to each other via a connection linkage such that each engaging member may rotate relative to each other engaging member.

In one example embodiment, one or more of a plurality of engaging members may comprise different widths or diameters. In one example embodiment, the width or diameter of each engaging member may decrease between a proximal end and a distal end of the obstruction removal system.

In one example embodiment, one or more engaging members may each have a plurality of struts defining a number of cells or openings when the one or more engaging members are expanded from a radially compressed configuration to a radially expanded configuration. The one or more engaging members may be composed of Nitinol or a similar material and may be laser cut from a tube or panel to achieve a desired profile shape.

In one example embodiment, one or more of the engaging members may have an asymmetric body shape and/or an asymmetric cell configuration between the proximal and distal portions of the engaging member. One or more of the engaging members may have a “tear drop” shape in which a distal end radially expands to a relatively large diameter and then begins to taper through the remaining proximal length to the proximal end of the engaging member.

In example embodiments in which each engaging member comprises a plurality of struts, the struts may be generally tapered in width so as to decrease in width toward the middle of the engaging member. Thus, the proximal struts may be tapered so that they are thicker near the center to provide more longitudinal rigidity when tension is applied without increasing radial force or tracking forces.

In one example embodiment, one or more of the engaging members may include one or more radiopaque markers, which may be disposed around at least a portion of one or more of the struts. The one or more of the struts may include one or more structural features or shapes to help retain the radiopaque marker in place, such as a “dog bone” strut shape including a central portion, a first projecting portion extending distally from the central portion, and a second projecting portion extending proximally from the central portion. The corners of the central portion may include projections such as bumps, nubs, or the like which act as stoppers to prevent the radiopaque marker from sliding off of the strut.

In one example embodiment, a mesh structure may be positioned within the engaging member or may be positioned outside and adjacent to the engaging member to function as a filter to trap smaller pieces of emboli. The mesh structure may be composed of a single braided wire or a plurality of braided wires.

In one example embodiment, one or more support wires may be included within the engaging member. The one or more support wires may support the engaging member in its expanded configuration.

In one example embodiment, an engagement means for capturing a thrombus includes a plurality of open cells and has a radially compressed configuration and a radially expanded configuration; with the radially expanded configuration forming a longitudinally asymmetric shape having a distal region that has a larger diameter than a middle region and a proximal region.

A method of removing an obstruction may comprise advancing one or more engaging members out of a delivery catheter and engaging an obstruction with the one or more engaging members.

A method of making an obstruction removal system may comprise forming one or more engaging members and connecting at least one of the one or more engaging members to an elongated member.

Another method of making an obstruction removal system may comprise providing an engaging member comprising a plurality of struts and applying a radiopaque material on or around one or more of the plurality of struts.

### DESCRIPTION OF EMBODIMENTS

Specific embodiments of the invention will now be described with reference to the accompanying drawings. This invention may, however, be embodied in many different forms and should not be construed as limited to the embodiments set forth herein; rather, these embodiments are provided so that this disclosure will be thorough and complete, and will fully convey the scope of the invention to those skilled in the art. The terminology used in the detailed description of the embodiments illustrated in the accompanying drawings is not intended to be limiting of the invention. In the drawings, like numbers refer to like elements. While different embodiments are described, features of each embodiment can be used interchangeably with other described embodiments. In other words, any of the features of each of the embodiments can be mixed and matched with each other, and embodiments should not necessarily be rigidly interpreted to only include the features shown or described.

For the purposes of the terminology described below, the terms clot, thrombus, embolus, and obstruction can be used synonymously. Though an obstruction removal device is described, the device can also be used to capture clots, thrombi, emboli, foreign bodies, or other matter. Engaging members on the device can engage clot, thrombus, embolus, foreign bodies, obstructions, or other matter.

For the purposes of this specification, use of the term “about” or “around” when referring to a value may be understood to mean within 5% of the stated value (either greater or lesser), inclusive.

The present invention may comprise an obstruction removal system comprising one or more engaging structures, such as one or more engaging members. An engaging structure, such as an engaging member, may be generally considered to be an expandable structure that, when in its expanded position, has a plurality of openings, cells, or spaces sized to allow clots or similar structures to engage and/or enter within an interior of the structure.

At least one of the engaging members may be connected, directly or indirectly, to an elongated member. Only one of the engaging members may be connected to the elongated member, such as a distal end of the elongated member, or multiple engaging members may be connected to the elongated member.

Where multiple engaging members are utilized, adjacent engaging members may be interconnected via a connection linkage such that each engaging member may rotate relative to other engaging members. Such a configuration may provide freedom of movement so as to augment the clot retrieval process.

Each engaging member may comprise a plurality of struts which define a plurality of cells or openings when the engaging member is expanded to a radially expanded configuration. The engaging member may have an asymmetric body shape and an asymmetric cell configuration between the proximal and distal portions of the engaging member. Thus, a first portion of the engaging member may have a different size and/or cell size/layout than a second portion of the engaging member (e.g., a distal half of the engaging member may have a different size and/or cell size/layout than a proximal half of the engaging member).

Where multiple engaging members are utilized, one or more of the engaging members may have a different size than one or more of the remaining engaging members. Thus, the present invention may include a plurality of engaging members each having different sizes (e.g., widths or diameters). The size of each engaging member may get smaller between the proximal and distal ends of the obstruction removal system. Pairs of similarly-sized engaging members may be grouped together, with each subsequent pair having a different size than the preceding pair.

The cells or openings of the engaging member may have a lower porosity (i.e., smaller cell sizes) at a distal portion of the engaging member and a generally higher porosity (i.e., larger cell sizes) at a middle portion of the engaging member. The generally higher porosity at the middle portion may aid in allowing clots to enter the engaging member for removal. The decreased porosity at the distal portion of the engaging member may prevent a clot from migrating through the engaging member, which can result in failure to retrieve.

The struts themselves may have portions with different widths. The struts may be generally tapered in width so as to decrease in width towards a middle of the engaging member (i.e., the struts may start off with a first width at a proximal portion of the engaging member and decrease in width towards the middle portion). Thus, the struts may be thicker near the center to provide more rigidity when tension is applied. Distal struts may be tapered in an opposite direction to the taper of the proximal struts.

One or more of the engaging members may include one or more radiopaque markers. Each radiopaque marker may comprise a wire wound around the strut, a wire coil, a wound ribbon, a sheet bent around the strut, a hypotube disposed around the strut, a bead, and the like. The radiopaque markers may be attached to the strut in various manners, such as but not limited to adhesives, welding, melting, and the like.

The struts may include structural features or shapes to aid in retaining the radiopaque marker thereon. One or more of the struts may include a “dog bone” shape having an area of increased width relative to adjacent areas of the strut on its proximal and distal ends. Projections on the ends of a central portion of the strut may prevent the radiopaque marker from sliding off proximally or distally.

The obstruction removal system may include a mesh structure which may function as a filter to trap smaller pieces of emboli to prevent loss during the clot retrieval process. The mesh structure may be at least partially within the engaging member or may be outside of and adjacent to the engaging member. The mesh structure may be comprised of a single braided wire or multiple braided wires formed in a wide range of different shapes.

The obstruction removal system may include one or more structures to provide support within one or more of the engaging members to aid in improving expansion and resist collapsing; particularly when traversing tortuous vasculatures. The one or more structures may comprise one or more support wires positioned within an interior of one or more of the engaging members. The one or more support wires may be heat set into a wide range of shapes, including a helical shape or a plurality of concentric rings.

Specific example embodiments are described further below. However, it should be understood that any of the features from any of the embodiments can be mixed and matched with each other in any combination. Hence, the present invention should not be restricted to only these embodiments, but any broader combination thereof.

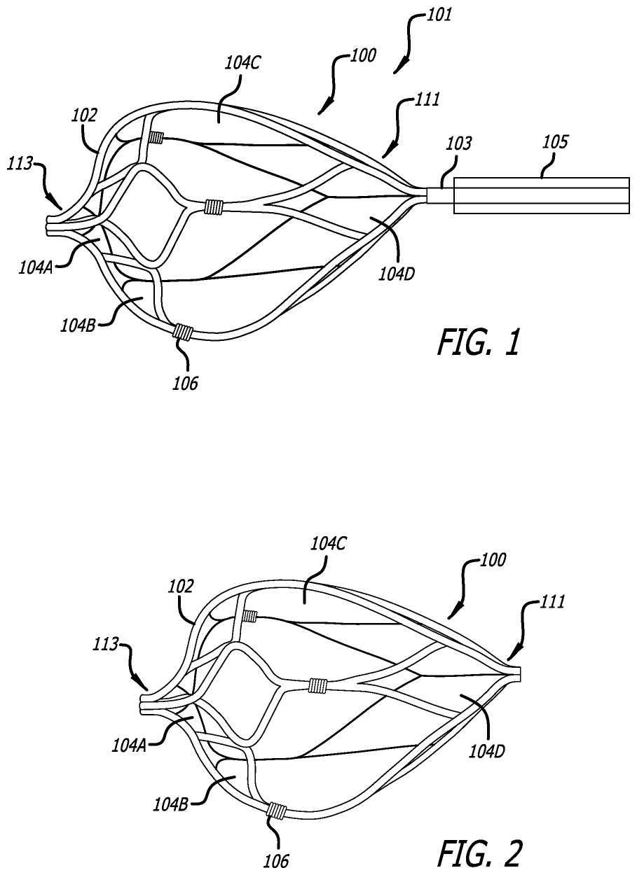

FIGS. 1-2 illustrate an engaging member 100 which may be used with various embodiments of an obstruction removal device 101. The systems and methods shown and/or described herein may be utilized with a wide range of obstruction removal devices 101, and thus the example embodiments shown in the figures should not be construed as limiting in scope. By way of example and without limitation, the systems and methods shown and/or described herein may be utilized in combination with the obstruction removal devices 101 shown and/or described in U.S. Pat. Nos. 10,729,455, 10,722,254, 10,709,466, 9,833,252, 9,770,251, 9,211,132, and U.S. Patent Publication No. 2019/0046210, all of which are hereby incorporated by reference in their entireties.

In one example embodiment such as shown in the figures, an obstruction removal device 101 may include one or more distal engaging members 100 which may engage thrombus or similar material which can accumulate within a vascular system. The engaging member 100 can be deployed adjacent to a thrombus or embolus and then proximally retracted to capture the thrombus or embolus within the engaging member 100. The engaging member 100 can then be further proximally withdrawn into a delivery device 105 and removed from the patient.

The systems and methods shown and/or described herein may be utilized with a wide range of delivery devices 105, and thus the example embodiments shown in the figures should not be construed as limiting in scope. By way of example and without limitation, example embodiments of a delivery device 105 may include various tubular medical delivery devices including but not limited to a catheter, a sheath, or a tubular jacket having a continuous passageway to allow passage of the obstruction removal device 101.

As shown in FIG. 1, an example embodiment of an obstruction removal device 101 can include an elongated member 103 which is connected at or near its distal end to the one or more engaging members 100. In some embodiments, the elongated member 103 can be a solid member. In other embodiments, the elongated member 103 may be tubular. For example, the elongated member 103 may include a lumen extending at least partially through its length. In some embodiments, the elongated member 103 can have a continuous lumen therethrough, thereby functioning like a catheter (e.g., can be used as a conduit for subsequently deployed items). The elongated member 103 may be considered as a pusher since it may be used to position the obstruction removal device 101 itself. The overlying delivery device 105 can be positioned over the elongated member 103 to allow both the elongated member 103 and the one or more engaging members 100 to be advanced out of and/or withdrawn into the delivery device 105.

Each engaging member 100 may have a collapsed configuration when sheathed within a delivery device 105 and may have an expanded configuration as shown in FIGS. 1-2 when unsheathed. Each engaging member 100 can be self-collapsible and self-expandable based on whether an external force is applied to constrain it (as would be the case when sheathed in a delivery device 105), or no constraining force is present (as would be the case when unsheathed). Each engaging member 100 can be composed of a shape-memory material (e.g., Nitinol) and can be heat set to impart an expansile shape memory. In this way, the engaging member(s) 100 may adopt an expansile shape when released from delivery device 105.

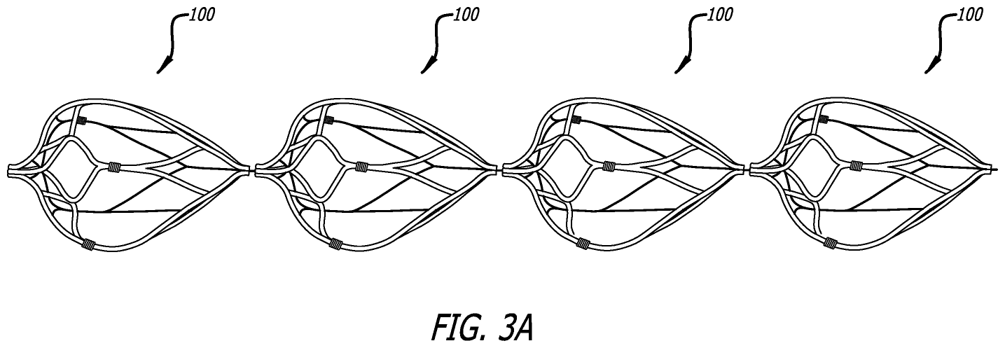

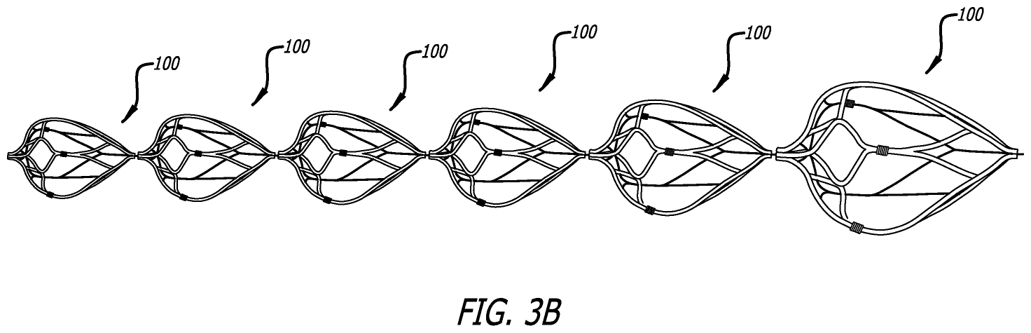

In one example, only one engaging member 100 may be located at and connected (directly or indirectly) to a distal end of elongated member 103. In another example, a plurality of engaging members 100 may be located at the distal end of the elongated member 103, as seen in FIG. 3A (note, in FIG. 3A that the elongated member 103 itself is not shown, but elongated member 103 may be linked to at least the most proximal engaging member 100 and, in some embodiments, may be linked to more than one engaging member 100).

The plurality of engaging members 100 may all be disposed over and connected to a wire or distal portion of the elongated member 103. Alternately, the proximal and distal ends of each engaging member 100 may be connected to each other via a connection linkage (e.g., an axle or pin with flared ends positioned within each engaging member 100). One advantage to this latter configuration is that each engaging member 100 may be capable of independent rotation relative to each other engaging member 100; this independent rotation can augment the clot retrieval procedure since each engaging member 100 has some freedom of movement.

In some example embodiments, the connection linkage can allow a degree of sliding movement for each engaging member 100 (e.g., the axle or pin with flared ends is slightly longer than a retention structure), thereby further augmenting the independence of movement of each engaging member 100. U.S. Pat. No. 9,211,132, hereby incorporated by reference in its entirety, has additional information on such linkage structures.

The one or more engaging members 100 may each have a plurality of struts 102 which define a number of cells or openings 104A-104D when the engaging member 100 is expanded from its radially compressed configuration to its radially expanded configuration. The engaging member 100 may be formed from Nitinol, or a similar material, and may be laser cut from a tube or panel to achieve the desired profile shape. Other materials and other cutting and/or machining processes would fit within the scope of the invention.

In one embodiment, the engaging member 100 may be configured to have a both an asymmetric body shape and an asymmetric cell configuration between the proximal portion and the distal portion of the engaging member 100 (i.e., longitudinally asymmetric). In other words, the a first portion of the engaging member 100 may not have the same size or cell size/layout as a second portion of the engaging member 100. In one example embodiment, a distal half of the engaging member 100 may have a different size or cell size/layout than a proximal half of the engaging member 100. In another example embodiment, a distal quarter length of the engaging member 100 may have a different size or cell size/layout than a proximal three quarters length of the engaging member 100. Various other configurations may be utilized in different embodiments to suit different applications.

FIGS. 3A-3B illustrate a plurality of engaging members 100 which are linked together. FIG. 3A illustrates an example embodiment having four engaging members 100. FIG. 3B illustrates an example embodiment having six engaging members 100. It should be appreciated that the number of engaging members 100 shown in the various example embodiments illustrated in the figures is not meant to be limiting in scope, as more or less engaging members 100 may be utilized in different embodiments to suit different applications. By way of example, some example embodiments may have less than three engaging members 100, five engaging members 100, or more than six engaging members 100.

In the example embodiment shown in the figures, the engaging members 100 are shown as being arranged linearly, with each of the engaging members 100 in an expanded configuration. It should be appreciated that, at times during delivery and/or deployment, the engaging members 100 may not be linearly arranged, such as when traversing a tortuous vessel.

FIG. 3A illustrates an example embodiment in which the engaging members 100 are each substantially the same size. However, in some embodiments, one or more of the engaging members 100 may be larger or smaller than one or more of the remaining engaging members 100. FIG. 3B illustrates an example embodiment in which the width or diameter of the engaging members 100 decreases between a proximal end and a distal end. In some embodiments, the reverse configuration may be utilized in which the width or diameter of the engaging members 100 increases between the proximal and distal ends. Thus, a proximal engaging member 100 may be larger in width or diameter than a distal engaging member 100, and a medial engaging member 100 between the proximal and distal engaging members 100 may be larger in width or diameter than the distal engaging member 100 and smaller in width or diameter than the proximal engaging member 100.

Continuing to reference FIG. 3B, it can be seen that the plurality of engaging members 100 may include pairs of engaging members 100 having a substantially similar width or diameter. In the illustrated example embodiment, the distal pair of engaging members 100 is larger in width or diameter than the medial pair of engaging members 100, and the medial pair of engaging members 100 is larger in width or diameter than the proximal pair of engaging members 100. Various other configurations of decreasing and/or increasing widths or diameters may be utilized in different embodiments. For example, in some embodiments, the width or diameter of the engaging members 100 may decrease and then increase, or vice versa.

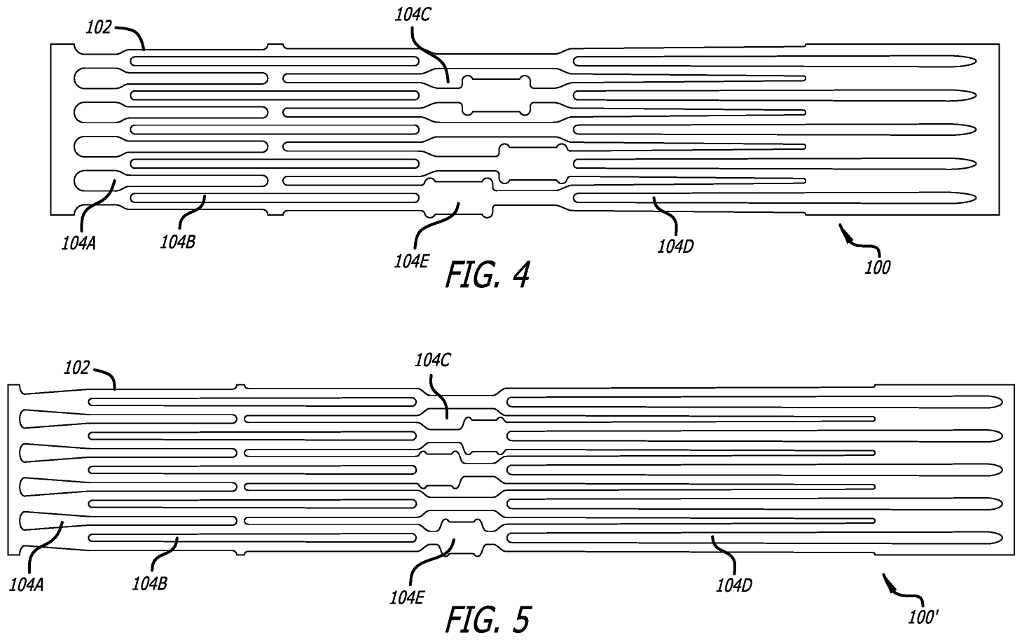

FIGS. 1 and 2 best illustrate the body shape and cells of an engaging member 100 in its radially expanded configuration, while FIG. 4 and the slightly modified design of FIG. 5 illustrate the engaging member 100 in a flattened configuration (i.e., as if the engaging member 100 was radially compressed, cut open, and laid flat in a plane). FIGS. 4-5 can be thought of as a shape achieved during the manufacturing processes of the engaging member 100 (e.g., a laser cut sheet) prior to the manufacturing steps taken to radially expand its shape.

As seen in these figures, the body shape of the engaging member 100 may have a generally “tear drop” shape in which its distal end 113 radially expands to a relatively large diameter and then begins to taper through the remaining proximal length to the proximal end 111 of the engaging member 100. In one example, the struts 102 of the engaging member 100 may radially expand to their largest distal diameter at about or around the first quarter length of the engaging member 100, starting from its distal end and the remaining three quarters length in the proximal direction tapers in diameter. The desired expanded shape can be achieved, for instance, by shape setting or heat setting the engaging member over a mandrel that forces it to the desired shape and diameter when expanded.

In one example embodiment, an engaging member 100 may have a length of about 3 mm when expanded and may reach a maximum radial diameter of about 2 mm at about 1 mm along its length in a proximal direction from its distal end, tapering with a decreased radial diameter proximally. In another example embodiment, the engaging member 100 may have a length of about 6 mm when expanded and may reach a maximum radial diameter of about 3 mm at about 2 mm along its length, tapering with a decreased radial diameter proximally. Thus, it should be appreciated that, in some embodiments, the maximum radial diameter of the engaging member 100 may be equal to between around 25%-75% of its length. In one example embodiment, the maximum radial diameter of the engaging member 100 may be equal to about 66% of the length of the engaging member 100. In another example embodiment, the maximum radial diameter of the engaging member may be equal to about 50% of the length of the engaging member 100.

As also seen in the figures, the cells 104C which comprise the open spaces of the engaging members 100, may be configured to have a generally lower porosity (i.e., smaller cell sizes) at the distal portion of the engaging member 100 and generally higher porosity (i.e., larger cell sizes) at the middle portion. In some embodiments, the generally higher porosity of the cells 104C may also be present at the proximal portion. The generally higher porosity at the middle portion may help allow clots to enter into the engaging member 100.

By decreasing porosity at the distal portion, a clot may be less likely to migrate through the engaging member 100 and more likely to be successfully retrieved. Additionally, this cell and strut pattern may allow for more flexibility at the middle of the engaging member 100 so that it can bend around curves in a vessel better while leaving the distal portion less effected or contorted in a manner that may negatively impact the ability to capture and release a clot.

A larger proximal porosity (i.e. larger proximal cells) may result in less strut area along the proximal section of the device, thereby lowering resistance to collapse when the engaging members 100 are resheathed. Furthermore, the generally tapered profile (e.g., increasing in overall diameter from a proximal end to a distal end of an engaging member 100) can allow for a smoother expansion and collapse of the engaging member 100 during delivery and/or deployment from a delivery device 105, thereby aiding in the clot retrieval procedure.

The above-described porosity can be adjusted by increasing or decreasing the number and/or size of each cell. In one example of the pattern of FIGS. 1-3, the engaging member 100 may have a length of about 3 mm. A first plurality of cells 104A (e.g., 4 cells) at a distal end of the engaging member 100 may have a cell size/diameter of about 0.5 mm. A second plurality of cells 104B (e.g., 4 cells) proximally adjacent to the first plurality of cells 104A may have a cell size/diameter of about 0.9 mm. A third plurality of cells 104C (e.g., 4 cells) proximally adjacent to the second plurality of cells 104B may have a cell size/diameter of about 2 mm. A fourth plurality of cells 104D (e.g., 4 cells) proximally adjacent to the second plurality of cells 104C may have a cell size/diameter of about 1.5 mm.

Any size/diameter values discussed herein are merely for exemplary purposes and should not be construed as limiting in scope. As described and shown in the figures, it can be seen that the first (distal-most) plurality of cells 104A may have a size/diameter that is less than a size/diameter of the second plurality of cells 104B, the second plurality of cells 104B may have a size/diameter that is less than a size/diameter of the third plurality of cells 104C, and the third plurality of cells 104C may have a size/diameter that is greater than the size/diameter of the fourth plurality of cells 104D. Thus, it can be seen that, starting from the distal end of the engaging member 100, the size/diameter of the cells 104A, 104B, 104C, 104D may first become greater, and then become smaller again at the proximal end.

Put differently as a function of length, in one example embodiment such as described above, the size/diameter of the first (distal-most) plurality of cells 104A may be equal to approximately 17% of the length of the engaging member 100, the size/diameter of the second plurality of cells 104B may be equal to approximately 30% of the length of the engaging member 100, the size/diameter of the third plurality of cells 104C may be equal to approximately 66% of the length of the engaging member 100, and the size/diameter of the fourth plurality of cells 104D may be equal to approximately 50% of the length of the engaging member 100.

With reference to each other, the first (distal-most) plurality of cells 104A may have a size/diameter that is approximately 55% of the size/diameter of the second plurality of cells 104B, the second plurality of cells 104B may have a size/diameter that is approximately 45% of the size/diameter of the third plurality of cells 104C, and the third plurality of cells 104C may have a size/diameter that is approximately 133% of the size/diameter of the fourth plurality of cells 104D in an example embodiment.

In another example of the pattern of FIGS. 1-3, the engaging member 100 may have a length of about 6 mm. A first plurality of cells 104A (e.g., 4 cells) at a distal end of the engaging member 100 may have a cell size/diameter of about 10 mm. A second plurality of cells 104B (e.g., 4 cells) proximally adjacent to the first plurality of cells 104A may have a cell size/diameter of about 15 mm. A third plurality of cells 104C (e.g., 4 cells) proximally adjacent to the second plurality of cells 104B may have a cell size/diameter of about 70 mm. A fourth plurality of cells 104D (e.g., 4 cells) proximally adjacent to the third plurality of cells 104C may have a cell size/diameter of about 20 mm.

Put differently as a function of length, in one example embodiment such as described above, the size/diameter of the first (distal-most) plurality of cells 104A may be equal to approximately 166% of the length of the engaging member 100, the size/diameter of the second plurality of cells 104B may be equal to approximately 250% of the length of the engaging member 100, the size/diameter of the third plurality of cells 104C may be equal to approximately 1,166% of the length of the engaging member 100, and the size/diameter of the fourth plurality of cells 104D may be equal to approximately 333% of the length of the engaging member 100.

Additionally, the porosity and performance characteristics of the engaging member 100 can be adjusted by increasing or decreasing the width of portions of each strut 102. For example, the struts may be generally tapered in width so they decrease in width toward the middle of the engaging member 100. Specifically, the struts 102 may start off with a first width at the proximal portion of the engaging member 100 that form cells 104D and decrease in width distally towards the middle portion of the engaging member 100, forming cells 104C. Similarly, the struts 102 may start off with a first width at the distal portion of the engaging member 100 forming cells 104A and decrease in width proximally towards the middle portion of the engaging member 100 forming cells 104C.

In other words, the proximal struts may be tapered so that they are thicker near the center of the hypotube to provide more longitudinal rigidity when tension is applied without increasing the radial force or tracking forces as much as if the strut 102 was thickened closer to the maximum outer diameter of the engaging member 100. Distal-most struts 102 may form a pattern that returns to a closed tube and are tapered in the opposite direction as proximal struts, as decreasing stiffness at this location allows the distal filter to occupy more intraluminal space as the struts 102 bend inwardly from the distal closed section and prevent the engaging member 100 from entirely opposing to the vessel wall. Large central apertures may allow a thrombus to integrate into the engaging member 100 and be captured by the distal end 113. Flexible proximal struts 102 may be held open in a parachute-shape as tension is applied, due to the disparity in radial and longitudinal stiffness between the proximal end 111 and distal end 113.

As seen in FIGS. 1-2, the engaging member 100 may include one or more radiopaque markers 106. These markers 106 may comprise a radiopaque material disposed around a portion of one or more of the struts 102. Various types of radiopaque materials known in the art may be utilized. For example and without limitation, the radiopaque material can be composed of tungsten, platinum-iridium with a balance of 90% platinum and the remainder iridium, gold, or similar materials.

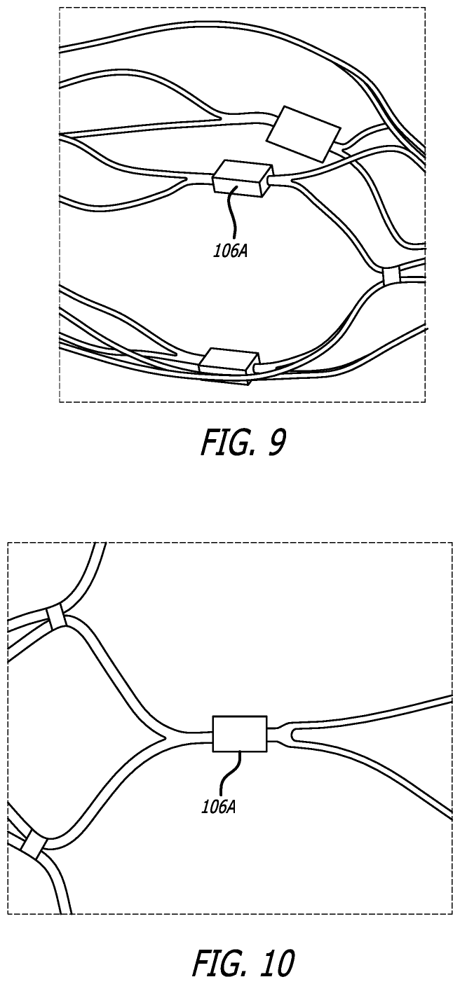

The radiopaque material can be in the form of a wire wound around the strut 102 such as shown in FIGS. 1-2, a wire coil, a wound ribbon, a sheet bent around the strut, or a hypotube disposed around the strut 102. FIGS. 9-10 illustrate an example embodiment of a marker 106A disposed around a strut 102. Optionally, any of these forms of radiopaque markers 106, 106A can be further attached via adhesive, welding, or melting to form a bead. In another example, the radiopaque material can comprise a solid member which is cut open (e.g., where a horizontal cut is placed to open up the material like a clamshell), and the member may then be mounted over the strut 102, and then adhesive or welding may be utilized to close the opening to secure the radiopaque material to the strut 102.

Non-wire markers, such as a sheet or hypotube may be designed with the least amount of material as possible to reduce the amount of bending of the strut 102 within the catheter. A marker made with a thin-walled sheet material can be wrapped around the strut so that the marker is one layer thick on the outside of the hypotube and two or more layers thick inside the hypotube, increasing the size of the marker without bending the strut away from the inner catheter liner excessively, which can cause shortening and plastic deformation of the struts.

The struts 102 may also include one or more structural features or shapes that help retain the radiopaque marker 106 in place. One structural example can be seen in the “dog bone” strut shape 104E seen in FIGS. 4 and 5. The shape 104E forms an area of increased width relative to adjacent areas of a strut 102 on its proximal and distal end. The enlarged shape 104E may further have a width that is larger at its proximal and distal ends relative to its middle portion. This may take the form of a generally rectangular shape with projections 104F such as “bumps” of increased width at its ends or a more tapered hourglass shape forming enlarged ends. The enlarged proximal and distal ends allow the radiopaque material, such as a radiopaque wire, to be wrapped around the middle portion and prevented from sliding off the shape 104E proximally or distally.

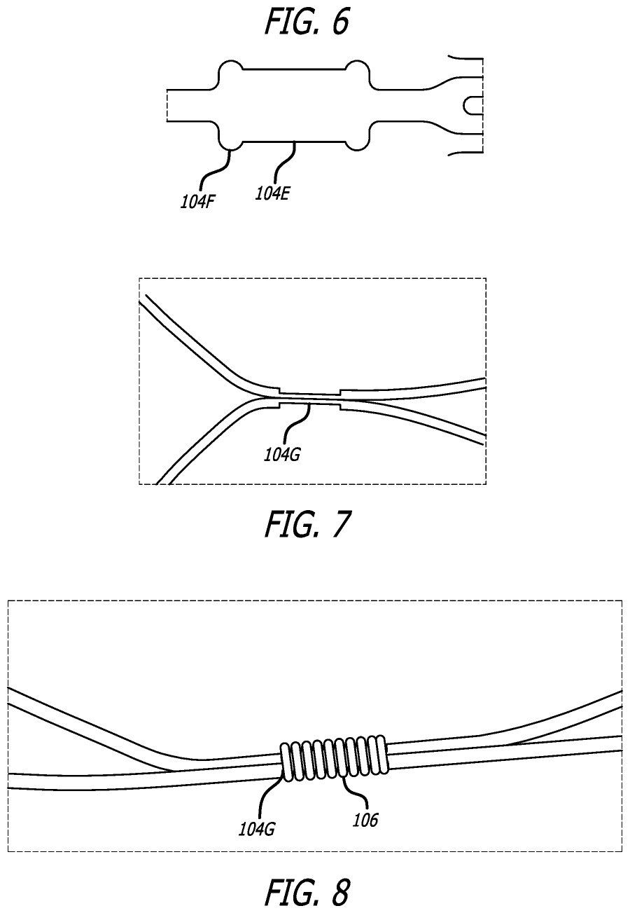

FIG. 6 is a closer view of an example embodiment of the strut shape 104E shown in FIGS. 4-5. As shown in FIG. 6, the strut shape 104E may comprise a central portion, a distal projection extending in a distal direction from a distal side of the central portion, and a proximal projection extending in a proximal direction from a proximal side of the central portion. Both of the distal and proximal projections may comprise a smaller width than that of the central portion from which they extend. As shown in FIG. 6, the corners of the central portion may include projections 104F such as bumps, nubs, or the like which act as stoppers to prevent the marker 106 from sliding off of the strut shape 104E.

One additional advantage to the increased width of the strut shape 104E is that it may show up as a relatively larger marker area under visualization techniques without the need to use a relatively larger amount of radiopaque material. In other words, the radiopaque material can be maintained as a thin layer over a greater surface area so that it visualizes well without the need for more radiopaque material that might otherwise increase the compressed size or other performance characteristics of the device. In one example, the strut shape 104E may have a length of about 0.2 mm, a middle width of about 0.005 mm, and end widths of about 0.007 mm.

Additionally, as seen in FIGS. 4-5, the strut shapes 104E can be radially offset from each other so that when the engaging member 100 is in a radially compressed configuration, the markers 106 do not contact each other. For example, two shapes 104E can be completely longitudinally offset from each other (e.g., the bottom two struts in FIG. 4) or can be positioned between two radially adjacent struts with no such structures 104E (e.g., the top three struts of FIG. 4).

Another structural shape example can be seen in FIGS. 7-8 in which an area 104G of the strut 102 may have a reduced diameter relative to proximally and distally adjacent portions of the strut 102. Radiopaque material, such as a radiopaque wire, may be wrapped around the reduced diameter area 104G, thereby maintaining a relatively small profile for the radiopaque marker 106.

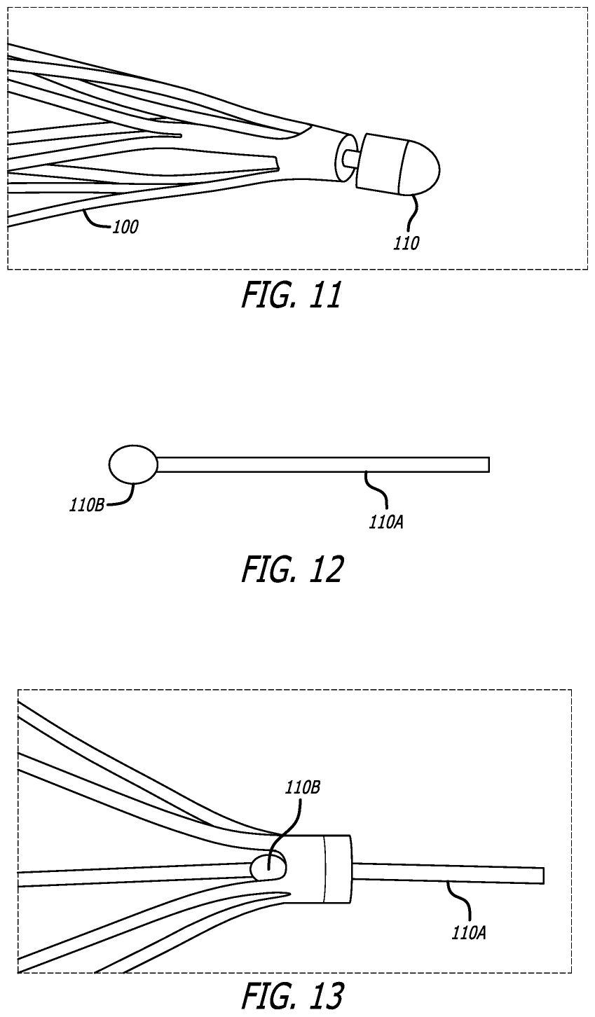

In the case of a single engaging member 100 or a distal engaging member 100 connected to a plurality of proximal engaging members 100, the distal tip may include a radiopaque marker 110, as seen in FIG. 11. The distal tip may comprise a radiopaque material, such as but not limited to platinum.

FIGS. 12-15 illustrate one example process for creating an example embodiment of a radiopaque marker 110. First, a metal wire 110A (e.g., stainless steel) may be created having an enlargement or ball 110B at one end such as shown in FIG. 12. For example, the wire can be about 0.040 inch to about 0.045 inch in length with an outer diameter of about 0.004 inch and an outer diameter of the enlargement 110B of about 0.0011 inch. The wire 110A may be advanced through a distal opening in the engaging member 100 such that the enlargement 110B is located proximally within the interior of the engaging member 100, as seen in FIG. 13.

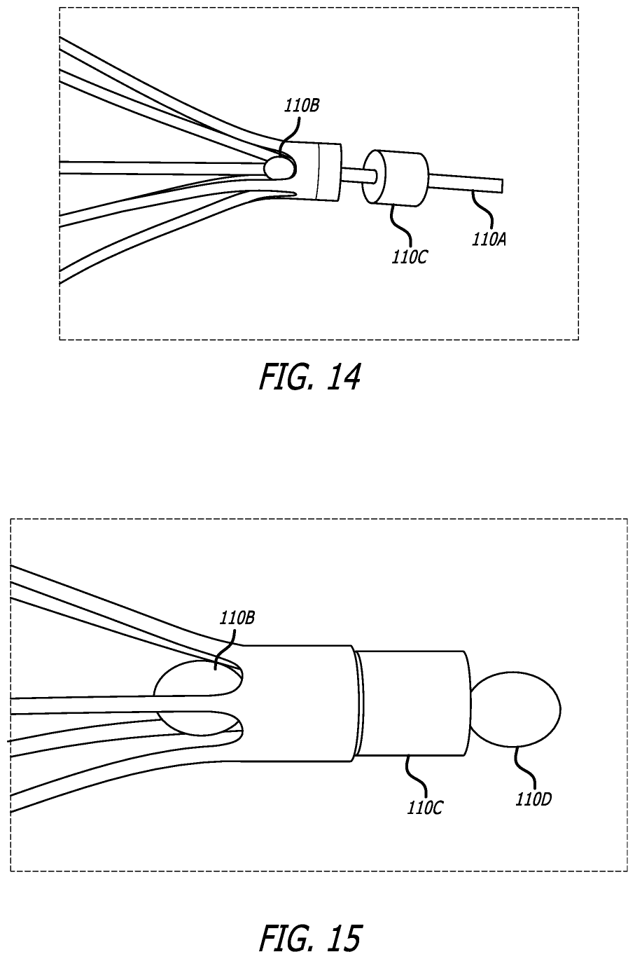

As seen in FIG. 14, a hollow cylinder or marker tube 110C may be advanced over the wire 110A. The marker tube 110C may be composed of radiopaque material, such as but not limited to a 90% platinum 10% iridium alloy, and in one example can be about 0.015 inch in outer diameter, 0.011 inch in length, have a wall thickness of 0.003 inch, and an inner diameter of about 0.006 inch. A UV glue fillet may be applied over the enlargement 110B and the distal face of the marker band 110C to create a smooth face.

Finally, a distal tip of the wire 110A may be welded to form a second, distal enlargement 110D on the distal side of the marker tube 110C, maintaining the marker tube 110C in position at the distal end of the engaging member 100, as seen in FIG. 15.

Once the nitinol marker band is welded onto the distal tip of the stentriever, there may be more than 0.005″ and less than 0.015″ of slack in the nitinol wire to allow for smooth tracking and articulation of the marker within the microcatheter. Additional UV glue may be applied to the marker tube 110C and distal enlargement 110D as necessary to help create a smooth surface.

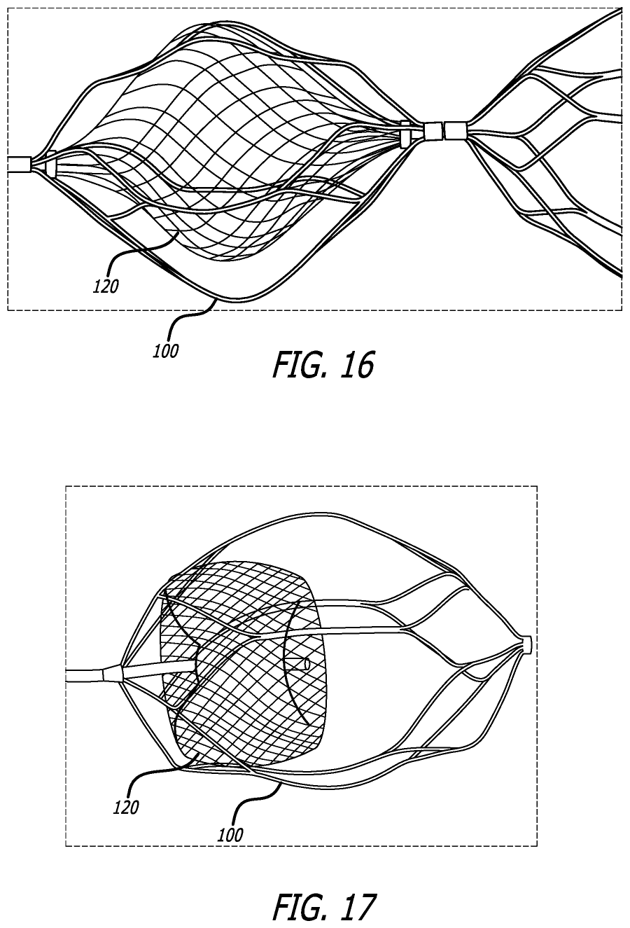

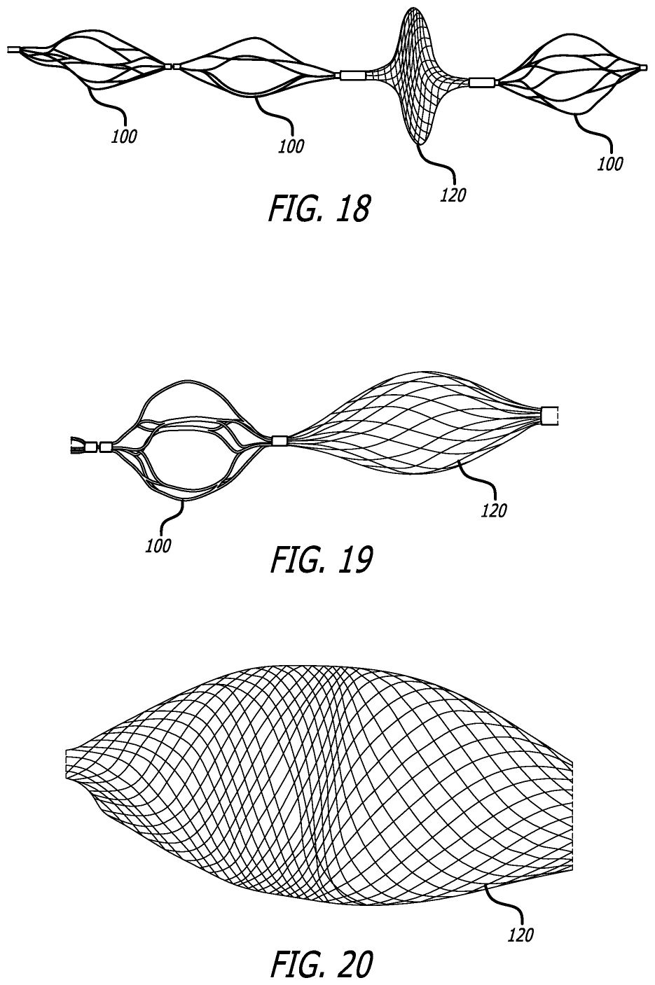

The obstruction removal device 101 may also be constructed with a mesh structure 120, either within the engaging member 100 such as shown in FIGS. 16-17 or outside and adjacent to the engaging member 100 such as shown in FIGS. 18-19. The mesh structure 120 may act as a further filter to trap smaller pieces of emboli, thereby helping to retain such smaller emboli pieces and minimizing the chance of these smaller pieces being lost during the clot retrieval procedure.

The mesh structure 120 may be composed of a single braided wire or a plurality of braided wires, such as Nitinol wires or drawn filled tubes having an outer layer of Nitinol and an inner core of radiopaque material. The mesh structure 120 can be shape set to expand to a desired three-dimensional shape, such as a spherical shape, a tapered oval shape, a disc shape, a cylinder shape, or various other shapes. The mesh structure 120 may be a completely enclosed shape (e.g., spherical) or may have an opening forming a concave, cup shape, or similar shape. In such a concave shape, it may be desirable to position the concave opening facing distally, so as to better capture emboli.

FIG. 16 illustrates an example embodiment of an engaging member 100 having an expandable, three-dimensional mesh structure 120 within it. The distal and proximal ends of the mesh structure 120 may be connected internally to the distal and proximal ends of the engaging member 100 so that both structures 100, 120 expand together. Hence, the mesh structure 120 may also assist the engaging member 100 in expansion and resisting collapse.

The mesh structure 120 of the embodiment of FIG. 16 is illustrated as having a longitudinally symmetrical shape (e.g., an oval with tapered ends), but alternately the mesh structure 120 may have a longitudinally asymmetrical shape, similar to the engaging member 100 shown in FIG. 1.

FIG. 17 illustrates another example embodiment in which a mesh structure 120 is positioned only in part of the interior of the engaging member 100, and specifically only the distal portion or distal half. However, in some embodiments, the mesh structure 120 may alternatively be positioned only in the proximal portion or proximal half.

In the example embodiment shown in FIG. 17, the mesh structure 120 may expand to a generally rounded cylindrical shape, through a variety of different shapes are possible. By positioning the mesh structure 120 distally, it may better catch emboli while leaving free the middle and proximal portions of the engaging member 100 to allow emboli to more easily enter the interior of the engaging member 100.

FIGS. 18-19 illustrate example embodiments in which a mesh structure 120 may be positioned externally with respect to an engaging member 100. By way of example, the mesh structure 120 may be positioned proximally of an engaging member 100 and optionally distally of another engaging member 100. The distal end of the mesh structure 120 can be connected to a proximal end of the engaging member 100, optionally in a manner that allows the two structures to rotate relative to each other.

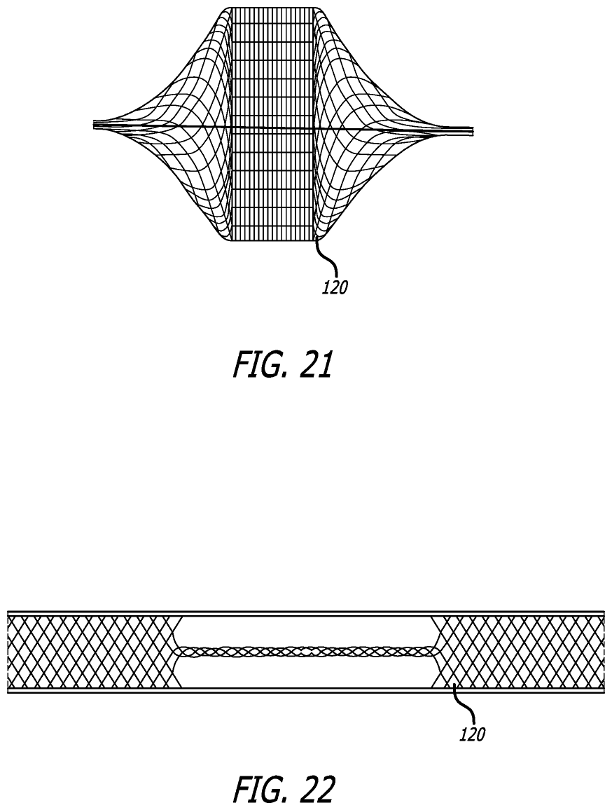

Additionally, the mesh structure 120 can have a uniform pore size/porosity along its length or different pore size after expansion. For example, a distal end may have a smaller pore size as seen in FIG. 20, or the middle portion may have a smaller pore size than the proximal or distal ends, as seen in FIG. 21.

In one example, the difference in pore size can be achieved by using different braiding patterns along the mesh structure 120. For example, the embodiment of FIGS. 20-21 may switch braiding patterns to increase the PPI (“picks per inch”) in different areas. Alternately, portions of the mesh structure 120 can be co-braided, as seen in FIG. 22, to reduce the porosity in different areas (e.g., the middle portion in the figure).

In another example, portions of the mesh structure 120 can be shape set to achieve different porosity with the same or different braid patterns, such as by creating more longitudinal expansion or contraction in areas when the mesh structure 120 is expanded.

In another example, portions of the mesh structure can be removed or trimmed to reduce porosity in desired areas. This may be especially helpful when creating a concave or cup shaped mesh structure 120.

The engaging member 100 may also include one or more structures to provide support within its structure to help improve expansions and resist it from collapsing, especially around highly curved areas of a vessel. As previously noted, any of the mesh structure 120 may provide this support.

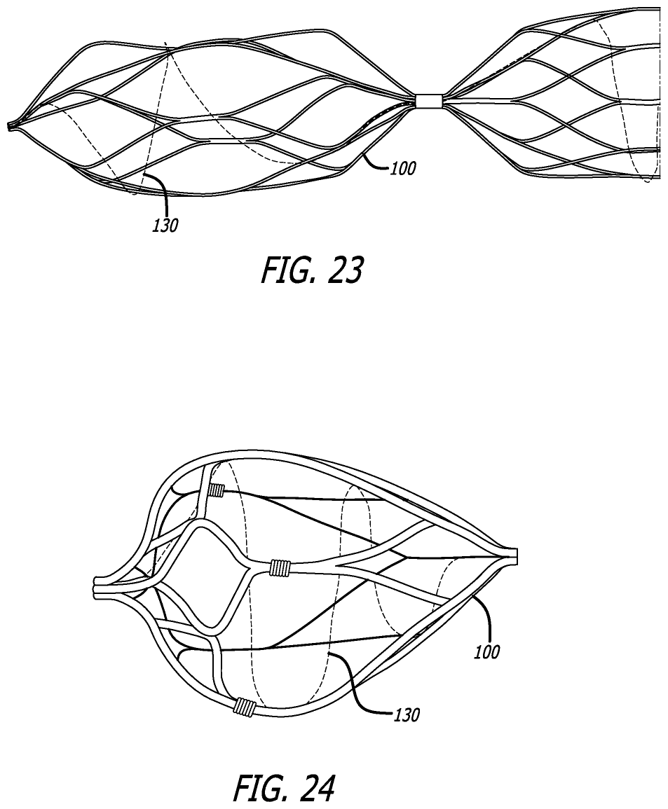

As shown in FIGS. 23-24, one or more support wires 130 may be included within the engaging member 100. Such one or more support wires 130 may be utilized instead of a mesh structure 120 or in addition to a mesh structure 120. The one or more support wires 130 may be heat set to form an expanded, three-dimensional shape that is similar or even larger in diameter than the interior of the engaging member 100, thereby helping to push it radially outwards. The shape of the one or more support wires 130 when expanded may vary in different embodiments and thus should not be construed as limited by the example embodiments shown in the figures. In one example embodiment, the one or more support wires 130 may expand into a helical or coiled shape.

The one or more support wires 130 may form a variety of different shape. For example, the wires 130 may form a generally helical shape as show in FIGS. 23-24. Alternately, the one or more support wires 130 may form one or more discrete circle shapes oriented perpendicularly to the axis of the engaging member 100.

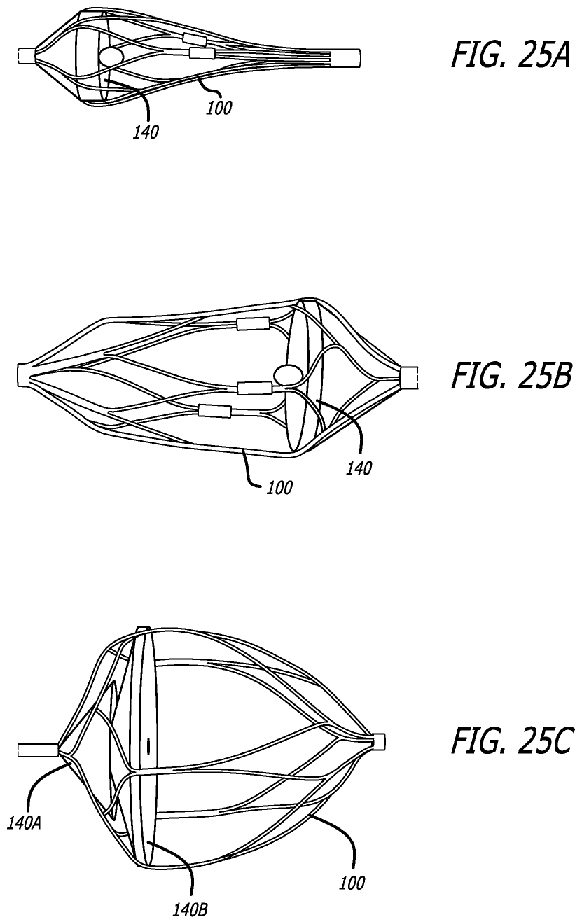

FIGS. 25A-25C illustrate a method of forming an example engaging member 100. As shown, one or more fixtures 140 may be utilized. First, wires may be braided or a tubular member such as a hypotube to form an initial shape. Subsequently, one or more fixtures 140 may be positioned within an interior of the initial tubular shape to form the asymmetrical shapes shown and described herein. As an example, a fixture 140 may be positioned within either end of the tubular member to form a taper prior to being heat set into a final expanded shape such as shown in FIGS. 25A and 25B.

FIG. 25C illustrates two separate fixtures 140A, 140B being utilized, with the first fixture 140A having a smaller diameter or width than the second fixture 140B. The use of multiple fixtures 140A, 140B may be desirable in situations in which a single fixture 140 would not fit within the tubular member. In the embodiment shown in FIG. 25C, the first fixture 140A may comprise a spacer plunger and the second fixture 140B may comprise an expansion plate.

### CLAUSES

Exemplary embodiments are set out in the following numbered clauses.

Clause 1. A method of removing an obstruction may comprise advancing one or more engaging members out of a delivery catheter; each of the one or more engaging members comprising a plurality of struts forming a plurality of open cells; the one or more engaging members having a radially compressed configuration and a radially expanded configuration; wherein the radially expanded configuration forms a longitudinally asymmetric shape having a distal region that has a larger diameter than a middle region and a proximal region; and, engaging an obstruction with the one or more engaging members.

Clause 2. A method of making an obstruction removal system may comprise forming one or more engaging members; each of the one or more engaging members comprising a plurality of struts and a plurality of open cells; the one or more engaging members having a radially expanded configuration and a radially compressed configuration; wherein the radially expanded configuration forms a longitudinally asymmetric shape having a distal region that has a larger diameter than a middle region and a proximal region; and, connecting at least one of the one or more engaging members to an elongated member.

Clause 3. A method of making an obstruction removal system may comprise providing an engaging member comprising a plurality of struts forming a plurality of open cells; at least one of the struts having a region of enlarged width forming a dog-bone shape; and applying a radiopaque material around a middle of the dog-bone shape.

Clause 4. A method of making an obstruction removal system may comprise providing an engaging member comprising a plurality of struts forming a plurality of open cells; at least one of the struts having a region of reduced width; and, applying a radiopaque material around the region of reduced width.

Clause 5. A method of making an obstruction removal system may comprise providing an engaging member comprising a plurality of struts forming a plurality of open cells; advancing a wire through a distal end of the engaging member; placing a radiopaque tube over the wire; and, creating an enlargement on a distal end of the wire.

Clause 6. A method of making an obstruction removal system may comprise providing an engaging member comprising a plurality of struts forming a plurality of open cells; and, connecting a mesh structure to the obstruction removal system or positioning a mesh structure near the obstruction removal system.

Clause 7. A method according to clause 6, wherein the mesh structure is connected at least partially within an interior of the engaging member.

Clause 8. A method according to clause 6, wherein the mesh structure is positioned externally to the engaging member.

Clause 9. A method of making an obstruction removal system may comprise providing an engaging comprising a plurality of struts forming a plurality of open cells; and, connecting a support wire within the obstruction removal system that is configured to expand against an interior of the engaging member.

It should be noted that any of the embodiment, features, or details of this specification can be used in connection with each other. In other words, while specific features may have been described separately, it is contemplated that any combination of these features can be combined with each other. Hence, this specification includes embodiments with any combination of the features described herein.

Although the invention has been described in terms of particular embodiments and applications, one of ordinary skill in the art, in light of this teaching, can generate additional embodiments and modifications without departing from the spirit of or exceeding the scope of the claimed invention. Accordingly, it is to be understood that the drawings and descriptions herein are proffered by way of example to facilitate comprehension of the invention and should not be construed to limit the scope thereof.

1. An obstruction removal system, comprising:
an elongated member; and,
an engaging member comprising a unitary, continuous body and having a plurality of open cells; the engaging member having a radially compressed configuration and a radially expanded configuration; wherein the radially expanded configuration forms a longitudinally asymmetric shape having a distal region that has a larger diameter than a middle region and a proximal region.

2. The obstruction removal system of claim 1, wherein the plurality of open cells have a lower porosity in the distal region than in the middle region.

3. The obstruction removal system of claim 1, wherein the engaging member comprises a plurality of struts.

4. The obstruction removal system of claim 3, wherein the plurality of struts taper to a smaller width, starting from either the proximal region or the distal region of the engaging member.

5. The obstruction removal system of claim 3, wherein at least one of the plurality of struts includes a region of reduced width and further comprising a radiopaque material disposed on or around the region of reduced width.

6. The obstruction removal system of claim 3, wherein at least one of the plurality of struts comprises a central portion, a proximal projection extending outwardly from a proximal end of the middle region, and a distal projection extending outwardly from a distal end of the middle region, and wherein the central portion is narrower than the proximal projection and the distal projection.

7. The obstruction removal system of claim 6, further comprising a radiopaque material disposed on or around the middle region of the at least one of the plurality of struts.

8. The obstruction removal system of claim 1, wherein the engaging member comprises a radiopaque distal tip.

9. The obstruction removal system of claim 8, wherein the radiopaque distal tip is comprised of a wire disposed around a distal end of the engaging member.

10. The obstruction removal system of claim 1, further comprising a mesh structure connected to the engaging member.

11. The obstruction removal system of claim 10, wherein the mesh structure is positioned within the engaging member.

12. The obstruction removal system of claim 10, wherein the mesh structure is positioned outside of and adjacent to the engaging member.

13. The obstruction removal system of claim 1, further comprising a support wire disposed within the engaging member.

14. An obstruction removal system, comprising:
an elongated member; and
a plurality of engaging members, wherein at least one of the plurality of engaging members is comprised of a plurality of struts having a plurality of open cells; the plurality of engaging members having a radially compressed configuration and a radially expanded configuration; wherein the radially expanded configuration forms a longitudinally asymmetric shape having a distal region that has a larger diameter than a middle region and a proximal region.

15. The obstruction removal system of claim 14, wherein the plurality of engaging members includes a distal engaging member and a proximal engaging member, and wherein a width of the distal engaging member is less than a width of the proximal engaging member.

16. The obstruction removal system of claim 15, wherein the plurality of engaging members includes a medial engaging member between the distal engaging member and the proximal engaging member, wherein a width of the medial engaging member is greater than the width of the distal engaging member, and wherein the width of the medial engaging member is less than the width of the proximal engaging member.

17. The obstruction removal system of claim 14, wherein the plurality of open cells have a lower porosity in the distal region than in the middle region.

18. The obstruction removal system of claim 14, further comprising a mesh structure connected to at least one of the plurality of engaging members.

19. The obstruction removal system of claim 14, further comprising a support wire disposed within at least one of the plurality of engaging members.

20. An obstruction removal system, comprising:
an elongated member;
an engagement means for capturing a thrombus including a plurality of open cells, wherein the engagement means has a radially compressed configuration and a radially expanded configuration; and,
wherein the radially expanded configuration forms a unitary, continuous longitudinally asymmetric shape having a distal region that has a larger diameter than a middle region and a proximal region.
